(19) United States
(12) Patent Application Publication
(10) Pub. No.: US 2025/0288730 A1
(43) Pub. Date: Sep. 18, 2025

(54) DEVICES, SYSTEMS AND METHODS USING CHLORINE DIOXIDE IN A CATHETER

(71) Applicant: Adapta Medical, Inc., Colorado Springs, CO (US)

(72) Inventor: Jamie Glen HOUSE, Colorado Springs, CO (US)

(21) Appl. No.: 19/078,142

(22) Filed: Mar. 12, 2025

Related U.S. Application Data
(60) Provisional application No. 63/564,115, filed on Mar. 12, 2024.

(51) Int. Cl. A61L 29/10; A61L 29/14; A61M 25/00

(52) U.S. Cl. CPC A61L 29/106; A61L 29/145; A61M 25/0045; A61L 2300/106; A61L 2300/452; A61L 2400/10; A61L 2420/06; A61M 2025/0046; A61M 2202/06; A61M 2205/0205

(57) ABSTRACT

A catheter system for draining a fluid from a bladder. The system includes a catheter combined with an amount of chlorine dioxide adapted to reduce infection in catheter associate urinary tract infections. The chlorine dioxide is adapted for use with at least one of a hydrophilic coating on an outer surface of the catheter, a wetting fluid that interacts with the hydrophilic coating and a lubricant in contact with the outer surface of the catheter.

### CROSS-REFERENCE TO RELATED APPLICATION

This application incorporates and claims the benefit of the filing date of U.S. Provisional Patent Application Ser. No. 63/564,115, entitled “DEVICES, SYSTEMS AND METHODS USING CHLORINE DIOXIDE LUBRICANTS” filed Mar. 12, 2024, the entirety of which is incorporated herein by reference.

### TECHNICAL FIELD

The present subject disclosure relates to use of chlorine dioxide based lubricants. More specifically, the present subject disclosure relates to urinary catheters using chlorine dioxide lubricants.

### BACKGROUND

Catheter-Associated Urinary Tract Infections (CAUTIs) create significant morbidity and mortality for individuals that require insertion of a urethral catheter to drain the bladder. Hospital-Acquired Urinary Tract Infections (HAUTIs) are common and are often due to antibiotic-resistant organisms found in the hospital. CAUTIs and HAUTIs contribute to recognized and excessive costs to the national healthcare expenditure. Conventional catheters are lubricated with a water-based gel or covered with a hydrophilic coating, which is combined with saline or a water-base wetting fluid. This creates a slick surface that decreases the resistance of the catheter during insertion and reduces discomfort to the patient.

Unfortunately, these conventional catheters are prone to germs entering the urinary tract and causing infection. Thus, there is a need for an infection prevention catheter solution that dramatically reduces the risk of catheter-associate urinary tract infections.

### SUMMARY

The present subject disclosure describes catheter devices, systems, and methods that address some of the shortcomings of conventional techniques.

The current subject disclosure presents novel catheter devices, systems, and methods having a lubricating gel that incorporates a chlorine dioxide chemical. Although the examples presented herein relate to urinary catheters, the use is not limited to such catheters and may be any particular device, system, or method which could benefit from the use and properties of chlorine dioxide as described herein. In the examples presented, the subject disclosure is described with respect to lubricating the external surface of urinary catheters. Although chlorine dioxide has been used in many industries to create a bactericidal environment that can eliminate pathogenic organisms, it has never been incorporated into a hydrophilic coating, a wetting fluid or a lubricating gel (or method for lubrication) for a urinary catheter. Integrating chlorine dioxide into these unique components, such as chlorine dioxide lubricating gel (or method for lubrication) during insertion of a catheter into the urethra and bladder will have the ability to dramatically decrease CAUTIs and HAUTIs worldwide. In some exemplary embodiments, the gel is internal to a protective sheath and protecting mechanisms as will be described in more detail later. The catheter can be protected by a sheath to prevent being touched by objects external to the catheter, the hydrophilic coating, the wetting fluid and gel.

In one exemplary embodiment, the present subject disclosure is a catheter system. The system includes a catheter having an outer surface; and a gel in contact with the outer surface of the catheter, wherein the gel includes chlorine dioxide.

In another exemplary embodiment, the present subject disclosure is a catheter system. The system includes a catheter combined with an amount of chlorine dioxide adapted to reduce infection in catheter associate urinary tract infections. The chlorine dioxide is adapted for use with at least one of a hydrophilic coating on an outer surface of the catheter, a wetting fluid that interacts with the hydrophilic coating and a lubricant in contact with the outer surface of the catheter. The catheter may include a sheath surrounding the various components; and the at least one of a hydrophilic coating, a wetting fluid and a lubricant gel contained within the sheath may include chlorine dioxide in a range of 0.05%-0.5% (w/v) of chlorine dioxide.

### BRIEF DESCRIPTION OF THE DRAWINGS

FIG. 1 shows a catheter assembly with a chlorine dioxide hydrophilic coating surface treatment disposed over the catheter tube, according to an exemplary embodiment of the present subject disclosure.

FIG. 2 shows a catheter assembly with a chlorine dioxide hydrophilic coating surface treatment disposed onto the catheter tube within a sheath, according to an exemplary embodiment of the present subject disclosure.

FIG. 3 shows a catheter assembly with a chlorine dioxide-containing lubricant coated catheter tube, according to an exemplary embodiment of the present subject disclosure.

FIG. 4 shows a catheter assembly with a chlorine dioxide-containing lubricant coated catheter tube within a sheath, according to an exemplary embodiment of the present subject disclosure.

FIG. 5 shows a catheter assembly with a catheter tube within a sheath containing chlorine dioxide wetting fluid, according to an exemplary embodiment of the present subject disclosure.

FIG. 6 shows a catheter assembly with a catheter tube removed from a packaging containing chlorine dioxide wetting fluid, according to an exemplary embodiment of the present subject disclosure.

FIG. 7 shows a catheter assembly with a chlorine dioxide-containing lubricant coated catheter tube within a urine collection bag, according to another exemplary embodiment of the present subject disclosure.

FIG. 8 shows a catheter assembly with a catheter tube within a urine collection bag containing chlorine dioxide wetting fluid, according to another exemplary embodiment of the present subject disclosure.

FIG. 9 shows a catheter assembly with a chlorine dioxide-containing lubricant coated catheter tube within a sheath and attached to a urine collection bag and, according to another exemplary embodiment of the present subject disclosure.

### DETAILED DESCRIPTION

The present subject disclosure addresses the shortcomings of conventional catheters, as discussed above and well known to one having ordinary skill in the art.

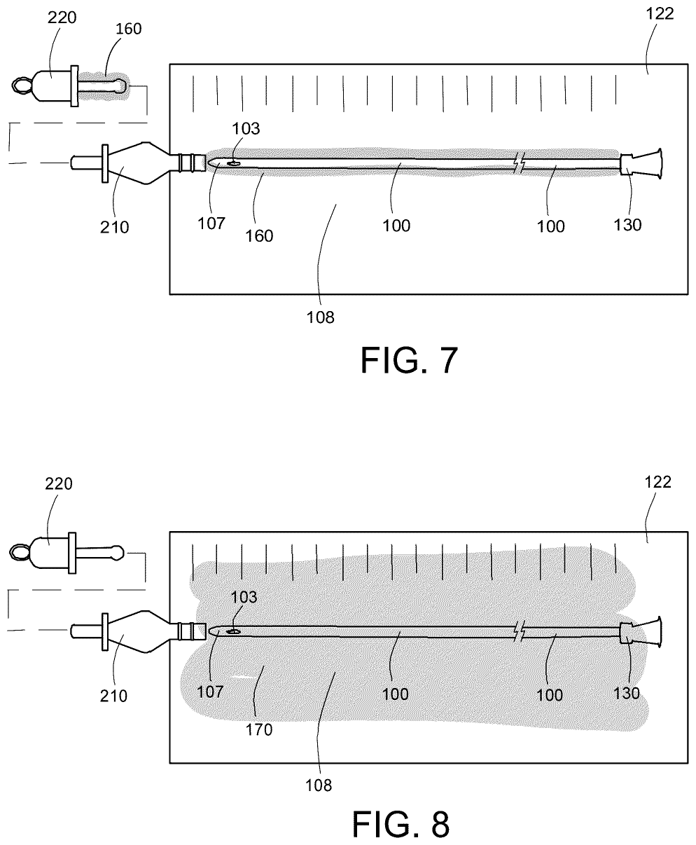

Current lubricating gels, hydrophilic coatings and wetting fluids do not have the ability to reduce or eliminate organisms that lead to the aforementioned urinary tract infections. It is known that closed-system (touchless) catheters 100 with an introducer tip 210 (similar to the catheter, introducer tip and sterile collection bag shown in FIGS. 7-8) housed within its own sterile protective cover 122. This arrangement decreases the risk of urinary tract infection by helping to bypass the bacteria in the outermost section of the urethra, however the use of the introducer tip 210 does not completely eliminate bacteria. Current urinary catheters have the possibility of touching the meatus prior to entering the urethra causing contamination and possibly infection. It has been shown that the first 1.5 cm of the urethra has the highest level of bacterial colonization. Another possible source of urinary tract infection is inserting a sterile lubricated catheter into the first part of the urethra that is colonized with bacteria. This bacteria is then pushed down the urethra and into the bladder leading to urinary tract infection.

When the catheter is inserted into the bladder, urine begins to flow into an eyelet 103 in the tip 107 of the catheter 100. It takes approximately 60 seconds or more for a full bladder to completely drain. During that time the catheter 100 is in direct contact with the urethra and bladder wall.

The present subject disclosure describes chlorine dioxide incorporated into lubricating gels 160, hydrophilic coatings 140 and wetting fluids 170, all of which cover and/or come into contact with an outer surface 105 of the catheter tube 100 that is inserted into the urethra and reaches the bladder. The use of the term catheter tube and catheter will be used interchangeably throughout this specification as 100. These various chlorine dioxide embedded components, i.e., the lubricating gels 160, hydrophilic coatings 140 and wetting fluids 170 expose the pathogenic organisms to a bactericidal or bacteriostatic chlorine dioxide compound. Chlorine dioxide is an effective compound capable of destroying bacteria, viruses, fungi and other cellular pathogens. These chlorine dioxide based compound components significantly reduce or eliminate urinary tract infections. The benefit of using chlorine dioxide is that it has been shown to be relatively benign to human tissue. Chlorine dioxide is used in an incomparable and disparate technology of purification of drinking water. Depending on the use for urinary tract infection treatment or for infection prophylaxis, the percentage of chlorine dioxide may vary. A predetermined concentration of chlorine dioxide that may be used according to this subject disclosure may be in the range of 0.005%-2% (w/v) by percent of solution in the total volume of solution which is safe for human use. Although not described in any detail, the use of choline dioxide as a gas on the outer surface of catheters may also be used.

Chlorine dioxide appears to be stable up to a pH of 10, but more active and bactericidal at the lower pH levels. The normal pH of the bladder is typically around 4.5 to 8.0, with an average of around 6.0. Various things such as diet and certain medical conditions can affect the pH. Therefore, chlorine dioxide is potentially more active and bactericidal in the normal urine environment, which may help prevent new tract infections. Several types of bacteria can increase the pH, such as Klebsiella, Pseudomonas, and Proteus. This condition can also be stone-forming and avoidance of infection with these bacteria is preferred.

Chlorine dioxide is a very strong oxidizer. This oxidizing activity is 2.5 times higher than diametric chlorine. It has a wide range of pH effective activity from 3-10. It is active against proteins in viruses, bacteria, and fungi. It's been shown to have bactericidal, fungicidal, sporicidal, tuberculocidal, and virucidal properties. Chlorine dioxide reacts with proteins and enzymes of the organisms' membranes leading to oxidative stress and cell death with inactivation of the microorganism. The bacterial cell loses its essential cellular components because of the permeability and destruction of the cell wall structure. Most organisms do not build a resistance to chlorine dioxide because of the level of oxidative load that it places on the cells. Chlorine dioxide also has an effect on ATPase activity reduction that causes injury to the metabolism of the bacteria and cell death.

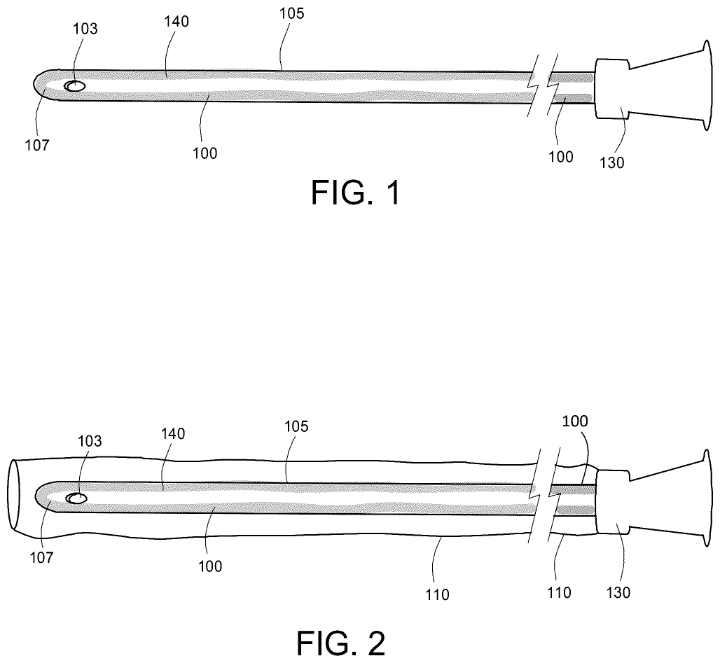

The present subject disclosure, as shown in FIGS. 1-9, includes several chlorine dioxide compound based components applied to an external surface of a catheter 100, such as a hydrophilic coating 140, a wetting fluid 170 and a lubricant gel 160.

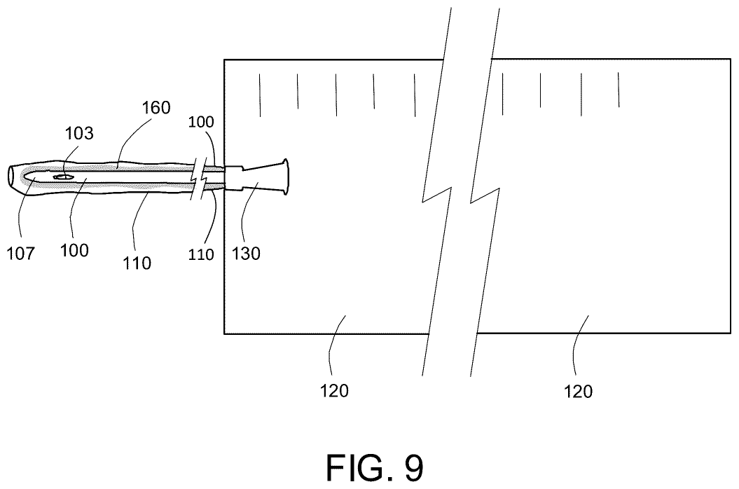

The catheter components, as shown in FIGS. 1-9, include a catheter tube 100 with varying French sizes (diameter) and length dependent on the particular product and use. The catheter 100 has a tip 107 with an eyelet 103 at one end and a funnel 130 at a second end. The catheter 100 can vary from several inches when used in a female product to 30 or more inches when extended to reach from the patient's bladder across the lap and into a toilet. Another component, shown in FIGS. 2, 4, 5 and 9, includes a sheath 110 or sleeve that covers all or part of the catheter 100. A urine collection bag 120, shown in FIG. 9, which may also be attached to the sheath 110 or sleeve at a funnel 130.

The lubricant may be any type of substance suitable for this subject disclosure. The lubricant described herein will be a chlorine dioxide included lubricating gel incorporating a water-based gel with a combination of dispersed chlorine dioxide into varying concentration ranging from 0.005%-15% (w/v) concentration of chlorine dioxide. The lubricating gel components are compatible with both ethylene oxide, irradiation (e-beam and gamma), and other methods of sterilization. Likewise, the hydrophilic coating surface treatment and wetting fluids used to improve lubricity and reduce friction during insertion and use may also be incorporated with similar concentration ranging from 0.005%-15% concentration of chlorine dioxide.

FIG. 1 illustrates a catheter 100 having a tip 107 with an eyelet 103 at a first end and a funnel 130 at a second end. A polymer or hydrophilic coating 140 is treated on an outer surface 105 of the catheter 100. The hydrophilic coating 140 is embedded with the chlorine dioxide at a predetermined concentration. The hydrophilic coating 140 binds to the outer surface 105 of the catheter 100. When the chlorine dioxide hydrophilic coating 140 is submersed in a fluid, such as water, the hydrophilic coating 140 absorbs and binds the water to the outer surface 105 of the catheter 100 making the outer surface 105 of the catheter 100 smooth and very slippery for easy entry into the urethra and into the bladder.

FIG. 2 shows a sheath 110 or sleeve covering all or part of the catheter 100 including the hydrophilic coating 140 treated on its outer surface 105. The sheath 100 allows the individual to grasp the catheter 100 without actually touching the external surface 105 of the catheter 100 causing contamination. The catheter 100 may then be inserted into the bladder. Ideally, the catheter 100 is completely covered with the external sheath 110 as this prevents the catheter 100 from being touched. The tip 107 can also be covered with an introducer tip 210 (as shown in FIGS. 7 and 8) or uncovered and bare as shown in FIG. 1.

To be most effective, the chlorine dioxide hydrophilic coating 140 should cover the proximal tip 107 (end that enters the bladder initially) and is able to provide lubrication upon contact with a wetting fluid 170 to allow the catheter 100 to easily slide into the urethra and eventually into the internal cavity of the bladder. As mentioned previously, the first 0.5 cm-1.5 cm of the urethra has the highest concentration of bacterial colonization. The chlorine dioxide hydrophilic coating 140 will be the first to enter this portion of the urethra and immediately begin to neutralize or destroy the pathogenic bacteria. The catheter 100 can be covered entirely with chlorine dioxide hydrophilic coating 140 during the drainage process, which usually takes approximately 60 seconds. During this time the chlorine dioxide hydrophilic coating 140 will interact to neutralize or destroy the pathogenic bacteria. Some of the hydrophilic coating 140 can reach the internal cavity of the bladder helping to further reduce the bacterial load and reduce or prevent urinary tract infections.

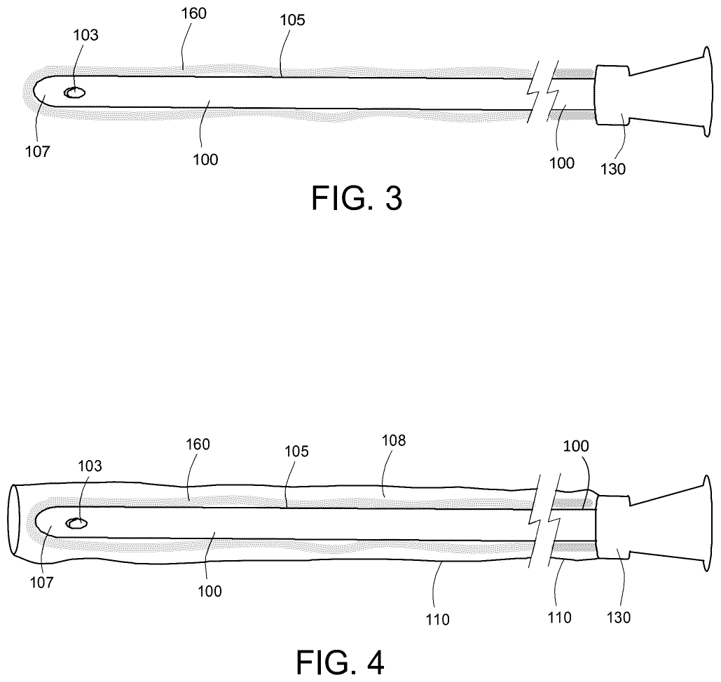

FIG. 3 depicts the catheter 100 is coated with the chlorine dioxide lubricating gel 160 on the outer surface 105 that initially enters the bladder. The lubricating gel 160 is applied to the outer surface 105 of the catheter 100. The lubricating gel 160 is combined with the chlorine dioxide at a predetermined concentration. The lubricating gel 160 makes the outer surface 105 of the catheter 100 smooth and very slippery for easy entry into the urethra and into the bladder.

FIG. 4 illustrates a sheath 110 or sleeve that covers all or part of the catheter 100. This allows the individual to grasp the sheath 110 over the catheter 100 without actually touching the external surface 105 of the catheter 100 causing contamination before the catheter 100 is inserted into the bladder. Ideally, the catheter 100 is completely covered with the external sheath 110 which prevents the catheter 100 from being touched. As will be described in more detail later, the tip 107 can be covered with an introducer tip (shown in FIGS. 7 and 8) or uncovered and bare such as shown in FIG. 3.

To be most effective, the chlorine dioxide lubricating gel 160 should cover the proximal tip 107 and is able to provide lubrication to allow the catheter 100 to easily slide into the urethra and eventually into the internal cavity of the bladder. As mentioned previously, the first 0.5 cm-1.5 cm of the urethra has the highest concentration of bacterial colonization. The chlorine dioxide gel 160 enters this portion of the urethra and immediately begins to neutralize or destroy the pathogenic bacteria. The entire catheter 100 can be covered entirely with the chlorine dioxide lubricating gel 160 during the 60 second drainage process, during which the chlorine dioxide lubricating gel 160 is interacting with the pathogenic bacteria. Some of the lubricating gel 160 can physically reach the internal cavity of the bladder helping to further reduce the bacterial load and reduce or prevent urinary tract infections. The chlorine dioxide lubricating gel 160 contacts the colonized bacteria that normally would be pushed into the bladder during insertion of the catheter and destroys it upon contact. That is, within seconds, the chlorine dioxide gel 160 begins to neutralize and or destroy the pathogenic bacteria. The length of catheter 100 either becomes partially coated with the lubricating gel 160 as it pushes through the lubricated gel 160 into the bladder or was previously entirely coated with the chlorine dioxide lubricating gel 160. During this process the lubricating gel 160 acts as a treatment against organisms that can lead to urinary tract infection. The portion of the catheter 100 that is covered with the chlorine dioxide lubricating gel 160 in the urethra can continue to treat the bacterial lining of the urethra and/or be carried into the urethra during the insertion process.

As shown in FIG. 4, the catheter 100 is coated with the chlorine dioxide lubricating gel 160. The chlorine dioxide lubricating gel 160 may also be placed in the space between the exterior surface 105 of the catheter 100 and an internal surface 108 of the protective sheath 110 that surrounds the catheter 100. The external sheath 110 prevents the individual user from touching the catheter 100. The sheath 100 also can act as a device that helps to grasp the slick, lubricated catheter 100 and insert into the bladder. Such situations may occur whether it's an intermittent catheter, or a lubricant used on a Foley catheter/indwelling catheter that remains in the bladder.

In order to make various embodiments of the chlorine dioxide lubricant of the present subject disclosure, a water-soluble lubricating gel may be used as a base. The exact composition of the gel is not necessary for the novel subject disclosure. Any suitable composition for a lubricating gel may be used according to this subject disclosure. The addition of the chlorine dioxide to the base lubricating gel 160 is one of the unique aspects of this subject disclosure. This combination of the chemicals in the gel make-up promotes that the even dispersion of chlorine dioxide throughout the gel and does not settle out as a solute. Various chemical percentages will be needed for different scenarios, but will all include the chlorine dioxide compound.

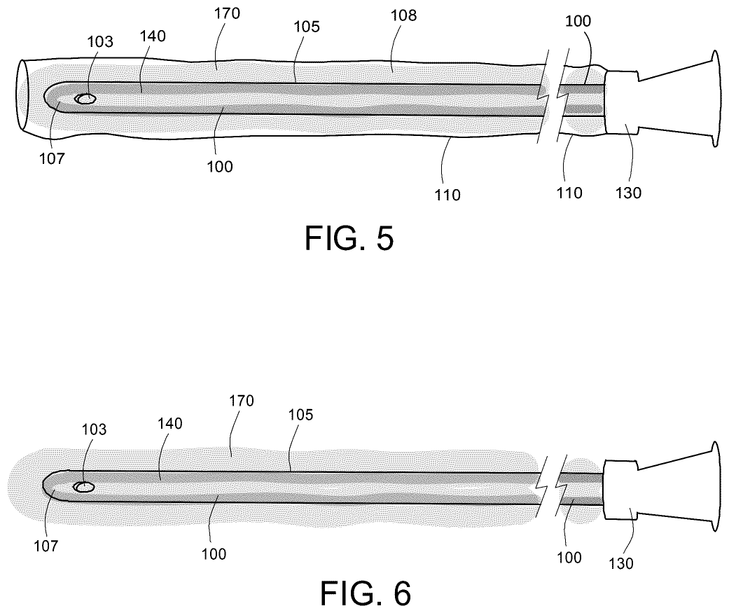

FIG. 5 shows another catheter assembly with a catheter tube 100 within a sheath 110 containing a chlorine dioxide containing wetting fluid 170 and hydrophilic coating 140, according to an exemplary embodiment of the present subject disclosure. As shown, the catheter 100 is covered from the tip 107 to its funnel 130 end surrounded by the sheath 110. The catheter 100 includes the hydrophilic coating 140 disposed along the outer surface 105 of the catheter 100. The sheath 110 covers the wetting fluid 170 containing chlorine dioxide and the hydrophilic coating 140 on the catheter 100 so as to prevent a user from touching the catheter 100.

FIG. 6 shows the catheter assembly with the catheter 100 removed from the sheath 110 packaging having the chlorine dioxide containing wetting fluid 170 and hydrophilic coating 140, according this present subject disclosure. As shown, the sheath 110 has been removed and the catheter 100 is ready for insertion. The wetting fluid 170 may be water, saline, a water based wetting fluid or other suitable wetting fluid. In use, when the chlorine dioxide hydrophilic coating 140 is submersed in the wetting fluid 170, the hydrophilic coating 140 will absorb and bind the water to the outer surface 105 of the catheter 100 making the catheter outer surface 105 smooth and very slippery for easy entry into the urethra and into the bladder.

FIG. 7 illustrates another exemplary embodiment in which the catheter 100 is packaged in a touchless closed system within a sterile protective cover 122 having an introducer tip 210. The sterile protective cover 122 may be used as a urine collection bag (as shown in FIG. 9). For example, in some urinary catheters 100 there is a need for the lubricating gel 160 to also be included and remain in a cap 220 that covers an introducer tip 210. In this situation the percentages of the chemicals and compounds may often require a higher viscosity gel. This can be accomplished by adjustments in the specific percentages of the chemicals and compounds. In other situations, the lubricating gel 160 is maintained within an external sheath 110 that is external to the catheter 100 (see FIG. 4). In these situations, the lubricating gel 160 often requires a lower viscosity and the chemical percentages could be adjusted to achieve this desired viscosity, as appreciated by one having ordinary skill in the art.

FIG. 8, similarly depicts, another exemplary embodiment in which the catheter 100 is packaged in a sterile protective cover 122 having an introducer tip 210 in which chlorine dioxide is combined with a wetting fluid 170. The chlorine dioxide infused wetting fluid 170 is spread out generously over the catheter 100. The chlorine dioxide wetting fluid 170 may be placed in the sterile protective cover 122 in a space between the exterior surface 105 of the catheter 100 and an internal surface 108 of the sterile protective cover 122 that surrounds the catheter 100. The sterile protective cover 122 prevents the individual user from touching the catheter 100. In use, the tip 107 of the catheter 100 can be pushed through the introducer tip 210 and inserted into the bladder.

FIG. 9 shows another embodiment including a catheter 100 enclosed within a sheath 110. A rear end funnel 130 of the catheter 100 is shown attached to a urine collection bag 120. The sheath 110 covering all or part of the catheter 100 includes a chlorine dioxide lubricating gel 160 on its outer surface 105. As mentioned before, the sheath 100 allows an individual to grasp the catheter 100 without actually touching the external surface 105 of the catheter 100 causing contamination. The catheter 100 may then be inserted into the bladder while the catheter 100 is completely covered with the external sheath 110 as this prevents the catheter 100 from being touched. In use, the lubricating gel 160 is able to provide lubrication upon contact to allow the catheter 100 to easily slide into the urethra and eventually into the internal cavity of the bladder. As shown, the catheter 100 can be covered entirely with the chlorine dioxide lubricating gel 160 during the urine drainage process to interact with the pathogenic bacteria. Likewise, some of the lubricating gel 160 can reach the internal cavity of the bladder helping to further reduce the bacterial load and reduce or prevent urinary tract infections.

The percentage of chlorine dioxide within the lubricating gel 160 also requires variability. In some situations, the lubricating gel 160 could be used in the form of treating a urinary tract infection. In other situations, it may be used chronically as a suppressant or prophylaxis against urinary tract infection.

The individual performing catheterization or the individual performing self-catheterization would either cover the proximal tip 107 of the catheter 100 with the lubricating gel 160 prior to insertion or use a catheter 100 that is previously coated with the chlorine dioxide lubricating gel 160.

The subject disclosure can be used with different technologies that require a lubrication gel, a hydrophilic coated device, a wetting fluid or lubricated device to enter a body cavity, thereby reducing a bacterial load. For example, during the insertion of a suprapubic tube or an enteral feeding tube nasally or through the abdomen, jejunum-tube or gastric-tube.

Other uses are also possible and within the purview of the present subject disclosure. As mentioned previously, the chlorine dioxide may be incorporated into the hydrophilic coating 140 or into the water or saline or other similar wetting fluid 170 that is either floating inside the sheath 110 or located in a sachet that is ruptured prior to use. See, for example, FIGS. 5, 6 and 8.

As described in this disclosure, the preferred chlorine dioxide is a stabilized chlorine dioxide. A preferred chlorine dioxide range for use in the hydrophilic coating 140, the lubricant gel 160 and/or the wetting fluids 170 may vary in the range from 0.005%-2.0% (w/v) by percent of solution in the total volume of solution. This same range in the hydrophilic coating 140, the lubricant gel 160 and/or the wetting fluids 170 could be used, but preferably in the range of 0.01%-0.8% (w/v), and perhaps more preferable in the range of 0.05-0.5% (w/v). These ranges fit within approved ranges set forth by the FDA for other ingestible uses.

The stabilized chlorine dioxide lubricant can be made by combining a solid chlorine dioxide tablet in a water-based solution or stabilized with the addition of a base and activated by acid environment. Other methods of making it are also within the purview of the present disclosure, as appreciated by one having ordinary skill in the art after consideration of the present disclosure.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed:

1. A catheter system for draining a fluid, the system comprising:
a catheter having an outer surface; and
a lubricant in contact with the outer surface of the catheter, wherein the lubricant includes chlorine dioxide.

2. The system in claim 1, wherein the chlorine dioxide is coated onto the outer surface of the catheter.

3. The system in claim 1, wherein the chlorine dioxide is incorporated into a hydrophilic coating on the outer surface of the catheter.

4. The system in claim 1, wherein the chlorine dioxide is incorporated into a wetting fluid to interact with a hydrophilic coating on the outer surface of the catheter.

5. The system in claim 4, wherein the wetting fluid comprises water.

6. The system in claim 4, wherein the wetting fluid comprises saline.

7. The system in claim 1, further comprising:
a sheath surrounding the catheter, the sheath containing the lubricant.

8. The system in claim 1, further comprising a collection bag positioned at one end of the catheter.

9. The system in claim 1, wherein the chlorine dioxide is used in a concentration of 0.005%-2.0% (w/v).

10. The system in claim 1, wherein the chlorine dioxide is used in a concentration of 0.01%-0.8% (w/v).

11. The system in claim 1, wherein the chlorine dioxide is used in a concentration of 0.05%-0.5% (w/v).

12. The system in claim 1, wherein the lubricant comprises a gel.

13. A catheter system for draining a fluid, the system comprising:
a catheter having an outer surface; and
a hydrophilic coating applied to an outer surface of the catheter, wherein chlorine dioxide is incorporated into the hydrophilic coating.

14. The system in claim 13, further comprising a lubricant in contact with the outer surface of the catheter.

15. The system in claim 13, wherein the lubricant includes chlorine dioxide.

16. The system in claim 13, wherein the chlorine dioxide is incorporated into a wetting fluid to interact with a hydrophilic coating on the outer surface of the catheter.

17. A catheter system for draining a fluid, the system comprising:
a catheter having an outer surface; and
a predetermined amount of chlorine dioxide is incorporated into at least one of:
a hydrophilic coating on the outer surface;
a wetting fluid that interacts with the hydrophilic coating; or
a lubricant in contact with the outer surface.

18. The system in claim 18, wherein the chlorine dioxide is used in a concentration of 0.005%-2.0% (w/v).

19. The system in claim 18, wherein the chlorine dioxide is used in a concentration of 0.05%-0.5% (w/v).

20. The system in claim 18, further comprising a sheath surrounding the catheter.
